(19) United States
(12) Patent Application Publication
(10) Pub. No.: US 2008/0119662 A1
(43) Pub. Date: May 22, 2008

(54) One Spot Synthesis of Citalopram from 5-Cyanophthalide

(75) Inventors: Ambati Narahari Babu, Karnataka (IN); Vuddamari Srinivas Goud, Karnataka (IN); Santosh Laxman Gaonkar, Karnataka (IN); Sulur G. Manjunatha, Karnataka (IN); Ashok Krishna Kulkarni, Karnataka (IN); Madhari A. Kulkarni, Karnataka (IN)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Jubilant Organosys Limted, Mysore (IN)

(21) Appl. No.: 10/589,387

(22) PCT Filed: Feb. 16, 2004
(86) PCT No.: PCT/IN04/00044

§ 371 Date: Dec. 18, 2007

(51) Int. Cl. C07D 307/87

(52) U.S. Cl. 549/467

(57) ABSTRACT

A process for one pot synthesis of citalopram is disclosed. The process comprises subjecting 5-cyano phthalide to Grignard reduction followed cyclization and followed by C-alkylation reaction to obtain citalopram without isolation and purification of any intermediates. In another embodiment, 5-cyano phthalide is subjected to sequential Grignard reactions followed by cyclization to obtain citalopram without isolation and purification of any intermediate stages.

### FIELD OF THE INVENTION

The present invention relates to the one pot synthesis of citalopram acid addition salts. In particular, the present invention relates to one pot synthesis of citalopram acid addition salts starting from 5-cyanophthalide without isolation and purification of any intermediate stages.

### BACKGROUND OF INVENTION

Citalopram and its pharmaceutically acceptable acid addition salts such as hydrobromide and hydrochloride have been described in U.S. Pat. No. 4,136,193. Citalopram is a potent antidepressant drug molecule with a few side effects. This has been in the market since a long time and its molecular structure is shown in Figure.-A.

The process of making citalopram lies in the art of sequential building of the molecule in a linear fashion. A number of processes have been disclosed in the prior art. For example, as described in U.S. Pat. No. 4,136,193 (Scheme-1), the process involves the reaction of 4-fluorophenylmagnesiumbromide with 5-bromo phthalide (2) to get 2-hydroxymethyl-4-bromo-4′-fluorobenzophenone(bromohydroxymethyl Ketone). This is reduced with lithium alluminium hydride and further cyclized in acidic media to get 5-bromo-1-(4-fluorophenyl)-1,3-dihydroisobnzofuran(5-bromo phthalane, (3). 5-Bromophthalane (3) is purified by high vacuum distillation and then reacted with cuprous cyanide in dimethyl formamide to get crude 5-cyano-1-(4-fluorophenyl)-1,3-dihydroisobnzofuran (5-cyano phthalane, (4), which is purified in ether to get the pure 5-cyano phthalane (4). 5-Cyanophthalane is alkylated with 3-N,N-dimethylaminopropylchloride in dimethylsulfoxide medium using a strong base like sodium hydride. Further by standard acid/base work up procedure followed by high vacuum distillation pure citalopram base (1) is isolated as an oil. The isolated citalopram oil is converted to its corresponding salts by conventional methods.

Another process disclosed in the same patent involves sequential Grignard reaction of 5-bromophthalide (2) with 4-fluorophenylmagnesiumbromide and 3-N,N-dimethylaminopropyl-magnesiumchloride in tetrahydrofuran (THF) medium to get the dihydroxy derivative(5) as an oil which is cyclised in aq.sulfuric acid followed by high vacuum distillation to get 1-(4-fluorophenyl)-1-(3-dimethylaminopropyl)-5-bromophthalane intermediate (6). The purified intermediate (6) is reacted with cuprous cyanide to get crude citalopram base. The crude citalopram base is purified by high vacuum distillation to obtain pure citalopram base (1) as an oil. Oily citalopram base is then converted to hydrobromide salt by conventional method followed by purification to get pharmaceutically

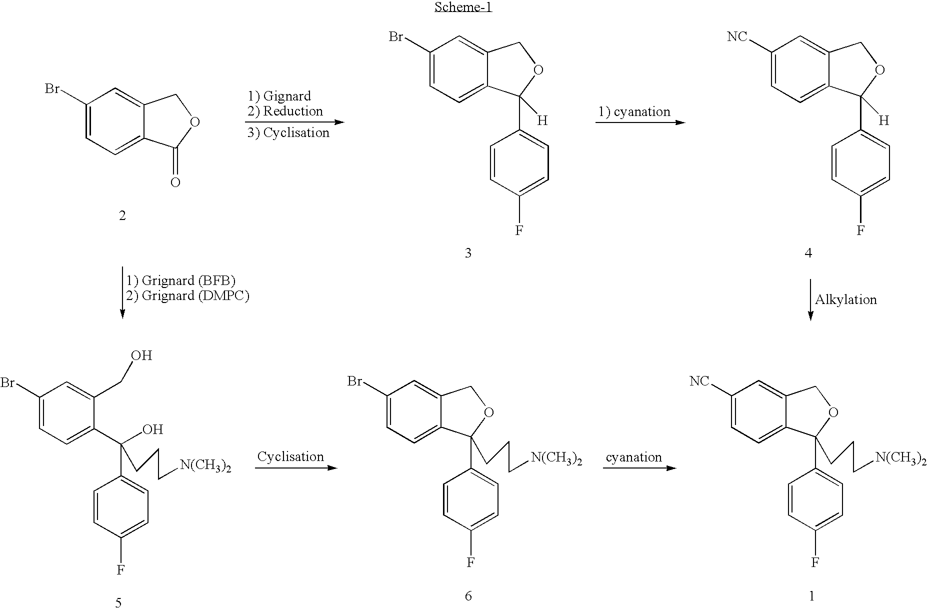

acceptable salt.

The disadvantages in the above two processes are a) THF is an expensive solvent used during the Grignard reaction and is not easily recoverable for the reuse and thus makes the process expensive from the commercial angle b) Intermediates are isolated by tedious work ups and some of the intermediates are purified by high vacuum distillation technique which are not easy to implement in the commercial level c) Citalopram base isolated as an oil, and is purified by high vacuum distillation at high temperature and in commercial plant is difficult to adapt the same.

Two processes for the preparation of citalopram starting from 5-cyanophthalide (7) is also known in the prior art and is shown in Scheme-2. The process as described in U.S. Pat. No. 4,650,884 involves sequential Grignard reaction of 5-cyanophthalide (7) with 4-fluoro-phenyl magnesium bromide and 3-N,N-dimethylaminopropylmagnesiumchloride in tetrahydrofuran (THF) medium to get the dihydroxy derivative (11) which is cyclised in aq.sulfuric acid to get citalopram base (1) as an oil. The oily base is reacted with anhydrous hydrogenbromide gas in acetone to get crude citalopram hydrobromide. Crude citalopram hydrobromide is further purified by repeated crystallization in different solvents to get the pharmaceutically acceptable grade of citalopram hydrobromide.

Another process involving the use of 5-cyanophthalide for the preparation of citalopram is described in WO 98/19511 (Scheme-2). According to the process described here a solution of 4-fluorophenyl magnesium bromide, generated in situ using 4-Fluorobromobenzene and magnesium in THF medium, is reacted with 5-cyanophthalide in THF medium to get 2-hydroxymethyl-4-cyano-(4′fluorophenyl) benzophenone, (CyanoHydroxymethylketone 8). Ethanol is added to the reaction mixture followed by the addition of excess sodium borohydride to get dihyroxy derivative (9) as an oil. The isolated dihydroxy derivative (9) is cyclized with aq. phosphoric acid to get 5-cyanophthalane [1-(4′-fluorophenyl)-1,3-dihydroisobenzofuran-5-carbonitrile (4). The crude 5-cyanophthalane (4) is crystallized from ethyl alcohol to get pure 5-cyanophthalane.

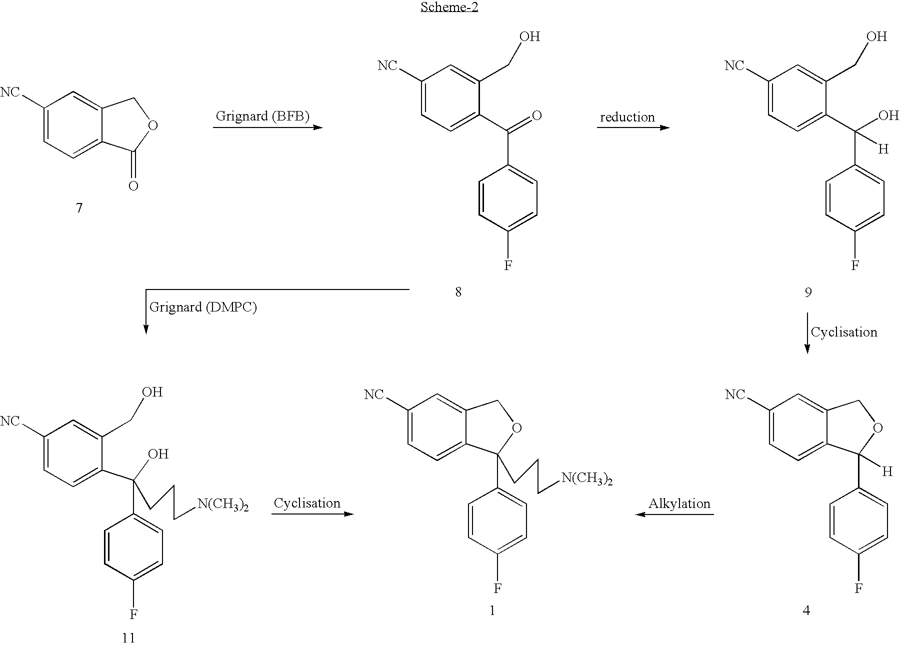

5-Cyanophthalane is alkylated with 3-N,N-dimethylaminopropylchloride in dimethoxyethane medium at −50° C. using a strong base (butyl lithium/diisopropylamine) and by standard workup Citalopram base (1) is isolated as an oil.

The major disadvantages in the above process are a) tetrahydrofuran is an expensive solvent and is used as a solvent during the Grignard reaction which is difficult to recover for recycle; b) the process involves lengthy and tedious procedures for the isolation and purification of intermediates for getting better quality of citalopram acid addition salts; c) butyl lithium, which is a strong base, highly reactive and moisture sensitive, is used in the process and is very difficult to handle in plant level because of the inherent hazardous nature of the material; d) the process demands a very low temperature, i.e., −50° C. Achieving and maintaining −50° C. in the commercial level is very difficult; e) anhydrous gaseous hydrogen bromide or hydrogen chloride, according to the process described here, is needed for preparing corresponding acid addition salts of citalopram. In the plant level, it is preferable to avoid handling of such gases because of their corrosive nature. Hence, these processes are not attractive for commercialization. Objects of the invention:

Accordingly, it is an object of the present invention to provide a process for one pot synthesis of citalopram.

It is another object of the present invention to provide a process for one pot synthesis of citalopram acid addition salts which minimizes or avoids the use of hazardous chemicals.

It is yet another object of the present invention to provide a process for one pot synthesis of citalopram acid addition salts which avoids the disadvantages of the prior art.

### SUMMARY OF THE INVENTION

The above and other objects of the present invention are achieved by providing one pot synthesis of citalopram starting from 5-cyano phthalide through single Grignard route, wherein 5-cyano phthalide is subjected to Grignard, reduction, cyclisation C-alkylation and followed by salt formation to obtain citalopram acid addition salts without isolation and purification of any intermediates (scheme-3). In another embodiment, 5-cyano phthalide is subjected to sequential Grignard reactions followed by cyclisation and salt formation to obtain citalopram acid addition salts without isolation and purification of any intermediate stages (scheme-4).

The present invention describes a very simple and efficient one pot process for the manufacture of citalopram acid addition salts. This process is easily adaptable to the commercial plant, starting from 5-cyanophthalide without isolation and purification of any intermediates.

### DETAILED DESCRIPTION OF THE INVENTION

Present invention describes a very simple procedure for the synthesis of citalopram acid addition salts starting from 5-cyanophthalide subjecting without isolation and purification of any intermediates (Scheme-3). As per the first part of the invention, a solution of 4-fluorophenyl magnesium bromide (1.0-1.4 moles), generated by reacting 4-fluorobromobenzene with magnesium and catalytic amount of iodine in tetrahydrofuran medium, is added to 5-cyanophthalide (1.0 moles) in an organic solvents below 10° C.

The molar ratio of 5-cyanophthalide with respect to 4-fluorophenyl magnesium bromide may be 1:1 to 1:1.4 and most preferred is 1:1.4. The organic solvent may be an ether such as diethyl ether, tetrahydrofuran; aliphatic halogenated solvents like methylene dichloride, ethylene dichloride, chloroform; aromatic hydrocarbons like benzene, toluene; aromatic halo carbons like chlorobenzene or combination of these solvents. The most preferred organic solvents are methylenedichloride, toluene or mixture thereof because the reactions at each stage can be worked up in such a way that the product in organic layer could be continuously taken for further stages to get corresponding citalopram acid addition salts.

After the completion of the Grignard reaction, the reaction mixture is quenched with aq ammonium chloride and the toluene layer containing the cyanohydroxymethylketone (8) is separated. Toluene layer is diluted with methanol followed by the addition of sodium borohydride (0.5 to 1.0 moles, preferably 0.5 molar equivalents) in lots over a period of an hour to get dihydroxy derivative (9). The reaction mixture is washed with water and the toluene layer is taken for cyclisation in the presence of acid. Organic acid, for example p.toluene sulfonic acid, benzene sulfonic acid, methane sulfonic acid is added to the toluene layer. The most preferred organic acid is p toluene sulfonic acid. In the present investigation, p-toluene sulfonic acid is used in catalytic amount (2%-10% w/w w.r.t 5-cyanophthalide). The reaction mixture is heated to reflux and water is removed by azeotropical distillation. The reaction mixture is then washed with aq. Sodium hydroxide to remove p.toluene sulfonic acid and then with water. Finally, the toluene layer contains 5-cyanophthalane is dried over sodium sulfate (anhydrous) and used as such for alkylation reaction with 3 N,N dimethylaminipropylchloride.

The dried toluene layer containing 5-cyanophthalane is added to a solution of a sodium, potassium salt of dimethysulfoxide, which is prepared by reacting strong base like sodium hydride or potassium tertiary butoxide in a mixture of dimethyl sulfoxide(DMSO) and toluene medium, at 20-25° C. followed by the addition of 3-N,N-dimethyl aminopropyl chloride as a solution in toluene. The reaction mixture is stirred at 20-25° C. for 1-3 hour and then quenched over ice cold water. The toluene layer is separated, washed with water and then extracted with 20% aqueous acid for example hydrochloric acid, hydrobromic acid, acetic acid, formic acid, preferably 20% aq. acetic acid solution. The aqueous acetic acid layer of citalopram may be used for the isolation of crystalline citalopram base as per the prior art procedures (EP 1346989 A1; WO 03/080590 A1). According to the present invention, the aqueous acetic acid layer is taken for base work up as follows:

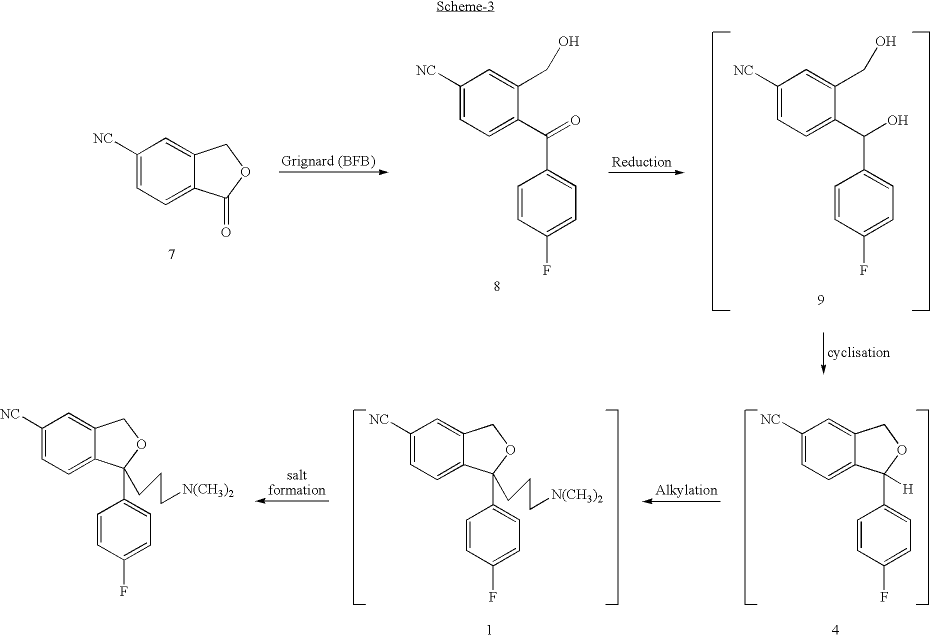

The aqueous acetic acid extract of citalopram base is cooled to 5-10° C. and the pH is adjusted to basic using a base at 5-10° C. Suitable base for adjusting the pH include liquor ammonia, sodium/potassium hydroxides and sodium/potassium carbonates but preferably a mild base such as ammonia is used. The liberated citalopram base is then extracted with suitable organic solvent like methylenechloride, ethylacetate, ether and toluene. The preferred solvent is toluene. The toluene layer is washed with water and dried over anhydrous sodium sulphate. The preferred way of isolating citalopram acid addition salts from the above toluene solution is treated with molar quantity of acid addition salts of week organic bases such as aniline, pyridine, picoline, pyrazine and pyrimidine. The preferred salts are pyridine hydrobromide and hydrochloride. The toluene solution is heated to 60-70° C. for 6-8 hours. The reaction mixture is cooled to 20-25° C. and the precipitated citalopram acid addition salts are filtered to get crude citalopram acid addition salts.

Another method for isolation of citalopram acid addition salts from the toluene layer is concentration under reduced pressure to get oily residue. The oily residue is dissolved in organic solvent groups selected from methanol, isopropyl alcohol, eththylacetate, acetonitrile and acetone or mixtures thereof. The preferred organic solvent is isopropyl alcohol and molar quantity of acid is added, acid group selected from hydrochloric acid, hydrobromic acid and oxalic acid at 5-10° C. over a period of 2 hours. The reaction mixture is cooled to 0-5° C. and the precipitated acid addition salt of citalopram is filtered. The salts can be further purified by dissolving in a solvent groups consisting of methanol, ethanol, isopropyl alcohol, acetone, acetonitrile, ethyl acetate and water or mixtures thereof to get pharmaceutically acceptable acid addition salts.

In the second part of the invention 5-cyanophthalide is reacted with 4-fluorophenyl-magnesiumbromide in THF/methylenechloride solvent mixture after completion of the reaction, then reaction mixture is treated at 0° C. to -5° C. with a solution of 3 N,N dimethylaminopropyl magnesium chloride (3 mole equivalent) in toluene/THF solvent mixture, which is prepared by the reaction of 3-N,N dimethylaminopropylchloride with magnesium in toluene/THF medium. After completion of the Grignard reaction, the reaction mixture is quenched with aq ammonium chloride and the organic layer containing the Dihydroxy (11) is separated. Organic layer is heated to 70-80° C. to distill off methylenechloride and THF. To the residual toluene layer aq. sulfuric acid (70%) is added and the mixture is heated to 70-80° C. for 3-4 h. After completion of the reaction, reaction mass is cooled and diluted with water then basified with liquor ammonia to separate the toluene layer. The toluene layer is then washed with Water and extracted with 20% aq. acetic acid. The aqueous acetic acid layer of citalopram may be used for the isolation of crystalline citalopram base as per the prior art procedures (EP 1346989 A1 ; WO 03/080590 A1 ). The aqueous acetic acid layer of citalopram is carried out base work up as disclosed in the first embodiment to get citalopram acid addition salts.

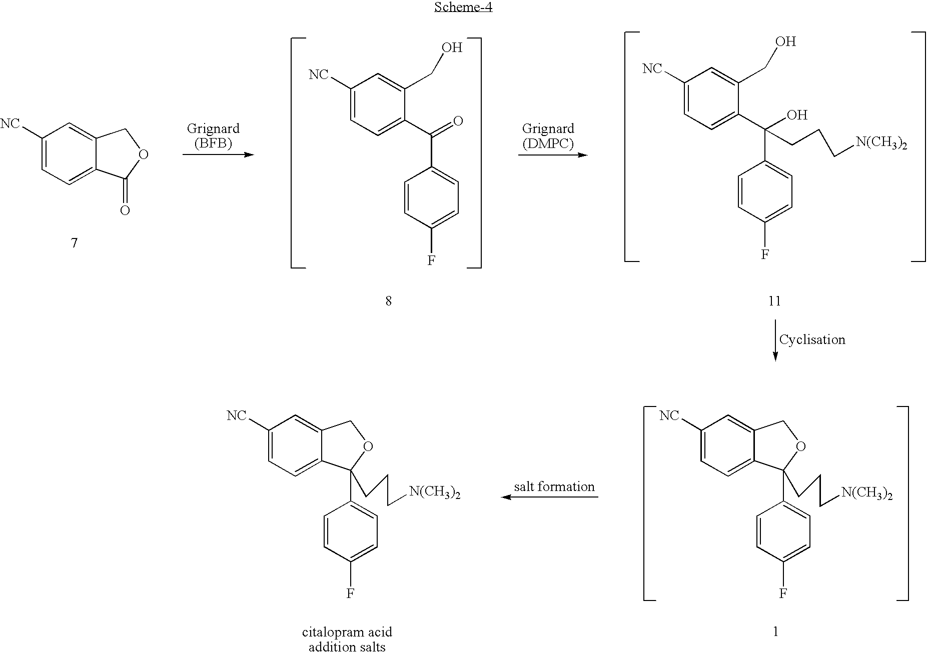

The major advantages of the present processes are a) a co-solvent such as toluene / MDC is used with tetrahydrofuran during the Grignard reaction. Apart from cost advantage and minimizing the risk involved in handling of tetrahydrofuran, the co-solvent assists in carrying the intermediates further to get citalopram without the isolation of any intermediates b) sodium borohydride (0.5 molar equivalent) is used in the reduction of hydroxyketone to dihydroxy derivative to improve the yield and quality c) Citalopram acid addition salts are isolated from the non aqueous medium like toluene using week acid addition salts of bases like pyridine hydrochloride and hydrobromide and thus avoiding use of corrosive anhydrous gases.

The following examples serve to further illustrate the present invention. In each, the citalopram salts purity is determined by HPLC and found to be in excess of 99%.

### EXAMPLE-1

### a) Process for the Preparation of Citalopram (by Single Grignard Method)

A solution of 4-fluorophenyl magnesium bromide, prepared from 153.33 g 4-flouro bromobenzene (0.876 moles) and 25.33 g magnesium turnings (1.055 moles) and Iodine (0.05 g.) in 300 ml of dry tetrahydrofuran was added to a suspension of 100 g 5-cyanophthalide (0.628 moles) in 900 ml dry toluene at −4 to −2° C. After the reaction was completed, the reaction mass was quenched with 100 ml 20% aqueous ammonium chloride solution. Toluene layer was separated and diluted with 100 ml of methanol. 12 g Sodium borohydride (0.324 moles) was added over a period of one hour at 10-15° C. and the same temperature was maintained for additional one hour. The reaction mass was quenched with 200 ml ice water and the toluene layer was separated. Toluene layer was washed with water (200 ml) and then 10 g of paratoluene sulphonic acid was added to toluene layer. The reaction mixture was heated to 80-85° C. and the temperature was maintained for additional 3 hours. After the completion of the reaction toluene layer was washed with aq. Sodium hydroxide solution (200 ml), water(200 ml) and dried over anhydrous sodium sulfate. The toluene solution was then added to a solution of 21 grams of sodium hydride dissolved in 400 ml of dimethyl sulfoxide and 500 ml toluene under nitrogen atmosphere at 20-25° C. To the resulting solution a solution of 3-N,N,-dimethylaminopropylchloride (53 g ) in 200 ml of toluene was added quickly at 20-25° C. The reaction mixture was stirred for 3 hrs at the same temperature. After completion the reaction the mixture was poured into ice water and the toluene layer was separated. The aqueous layer was extracted again with toluene. The combined toluene phase was extracted with 200 ml 20% aqueous acetic acid (40 ml acetic acid and 160 ml water). The aq. acid extract was cooled to 5-10° C. and the pH was adjusted to basic using liquor ammonia (85 ml) at 5-10° C. and extracted with toluene 3×300 ml. The toluene layer was washed with water and dried over anhydrous sodium sulphate. The toluene layer was treated with carbon (10 g) and filtered. The filtrate toluene is subjected to salt formation as per following methods.

### a) Preparation of Crude Citalopram Hydrobromide (Pyridine Hydrobromide)

Pyridine hydrobromide (37 gm) was added to the above toluene layer (Example-1) heated to 60-70° C. for 6-8 hours. The reaction mass was cooled to 20-25° C. then stirred for 4-hours and cooled to 10° C. The citalopram hydrobromide salt so precipitated was separated by filtration followed by washing with chilled toluene (200 ml)

### Dry weight=90-100 gm

### b) Preparation of Crude Citalopram Hydrochloride (Pyridine Hydrochloride)

Pyridine hydrochloride (27 gm) was added to the above toluene layer (Example-1) heated to 60-70° C. for 12-16 hours . The reaction mass was cooled to 20-25° C. then stirred for 4-hours and cooled to 10° C. The citalopram hydrobromide salt so precipitated was separated by filtration followed by washing with chilled toluene (200 ml)

### Dry weight=65-70 gm

### c) Preparation of Crude Citalopram Hydrobromide (Aq. Hydrobromic Acid)

The above toluene layer (Example 1) was concentrated under reduced pressure to get oily citalopram base was dissolved in 600 ml of isopropyl alcohol followed by the addition of 47% hydrobromic acid (30-35 ml). The reaction mass was then stirred for 4 hours at 25-30° C. and cooled to 10° C. The citalopram hydrobromide salt so precipitated was separated by filtration followed by washing with chilled Isopropyl alcohol (200 ml)

### Dry weight=90-100 gm

### d) Preparation of Crude Citalopram Hydrochloride (Aq. Hydrochloric Acid)

The above toluene layer (Example 1) was concentrated under reduced pressure to get oily citalopram base was dissolved in 600 ml of isopropyl alcohol followed by addition of 36% hydrochloric acid (30-35 ml) was added. The reaction mass was then stirred for 4 hours at 25-30° C. and cooled to 10° C. The citalopram hydrochloride salt so precipitated was separated by filtration followed by washing with chilled Isopropyl alcohol (200 ml).

### Dry weight=80-90 gm

### EXAMPLE-2

### Process for the Preparation of Citalopram (by Double Grignard Method)

A solution of 4-fluorophenyl magnesium bromides prepared from 153.33 g 4-flouro bromobenzene (0.876 moles) and 25.33 g magnesium turnings (1.055 moles) and Iodine (0.05 gm) in dry 300 ml tetrahydrofuran was added to a suspension of 100 g 5-cyanophthalide (0.628 moles) in 900 ml dry methylene dichloride at −4 to −2° C. After the completion of the reaction a solution of 3-N,N dimethylaminopropyl magnesiumchloride in toluen/THF mixture [generated in situ by reacting 175 g 3-N,N dimethylamiopropyl chloride(1.446 mole) in 350 ml toluene with 41.6 gm magnesium turnings(1.733 moles) and iodine( 0.05 g) in dry 75 ml tetrahydrofuran and dibromoethane] was added between 0-50C. The reaction mass was then maintained at −5 to 0° C. for 3-4 hours. After completion of the reaction, the reaction mass was quenched with 200 ml 20% aqueous ammonium chloride solution. The toluene layer was separated and washed with 200 ml water. Methylene dichloride and THF was distilled. 189 g sulphuric acid and 60 ml of water was added to the toluene layer and heated to 85-90° C. The same temperature was maintained for additional 4-5 hours. After completion of the reaction the reaction mass was diluted with 200 ml water and the pH was adjusted to basic with liquor ammonia below 10-15° C. The toluene layer was separated, washed with 200 ml water and extracted with 400 ml 20% acetic acid (80 ml acetic acid and 320 ml water). The aq. acid extract was cooled to 5-10° C. and the pH was adjusted to 8.5 to 9.0 using liquor ammonia (85 ml) at 5-10° C. and extracted with toluene 3×600 ml. The toluene layer was washed with water, dried over anhydrous sodium sulphate. The dried toluene layer was treated with carbon (10 g) and filtered. The filtrate toluene was subjected to salt formation in accordance with the following methods:

### a) Preparation of Crude Citalopram Hydrobromide (Pyridine Hydrobromide)

Pyridine hydrobromide (78 gm) was added to the above toluene layer (Example-1) and heated to 60-70° C. for 6-8 hours. The reaction mass was cooled to 20-25° C. then stirred for 4-hours and cooled to 10° C. The citalopram hydrobromide salt so precipitated was separated by filtration followed by washing with chilled toluene (200 ml)

### Dry weight=165-170 gm

### b) Preparation of Crude Citalopram Hydrochloride (Pyridine Hydrochloride)

Pyridine hydrochloride (57 gm) was added to the above toluene layer (Example-1) heated to 60-70° C. for 12-16 hours . The reaction mass was cooled to 20-25° C. then stirred for 4-hours and cooled to 10° C. The citalopram hydrobromide salt so precipitated was separated by filtration followed by washing with chilled toluene (200 ml)

### Dry weight=120-130 gm

### c) Preparation of Crude Citalopram Hydrobromide (Aq. Hydrobromic Acid)

The above toluene layer (Example 1) was concentrated under reduced pressure to get oily citalopram base was dissolved in 1000 ml of isopropyl alcohol followed by the addition of 47% hydrobromic acid (45-50 ml). The reaction mass was then stirred for 4 hours at 25-30° C. and cooled to 10° C. The citalopram hydrobromide salt so precipitated was separated by filtration followed by washing with chilled Isopropyl alcohol (300 ml)

### Dry weight=150-160 gm

### d) Preparation of Crude Citalopram Hydrochloride (Aq. Hydrochloric Acid)

The above toluene layer (Example 1) was concentrated under reduced pressure to get oily citalopram base was dissolved in 900 ml of isopropyl alcohol followed by addition of 36% hydrochloric acid (45-50 ml) was added. The reaction mass was then stirred for 4 hours at 25-30° C. and cooled to 10° C. The citalopram hydrochloride salt so precipitated was separated by filtration followed by washing with chilled Isopropyl alcohol (300 ml).

### Dry weight=120-130 gm

### EXAMPLE-3

### Process for Purification of Citalopram Hydrobromide/Hydrochloride

### a) Citalopram Hydrobromide (Methanol/Isopropyl Alcohol)

Citalopram hydrobromide (100 gm) was dissolved in methanol (200 ml) at 55-60° C. and then treated with carbon and filtered and washed with methanol (100 ml) The clear filtrate was diluted with isopropyl alcohol(600 ml). The resulting solution was cooled to 5-10° C., to obtain a crystallized product. The crystallized product was filtered and washed with chilled isopropyl alcohol to get pure citalopram hydrobromide

### Dry weight=85-90 gm

### e) Citalopram Hydrobromide (Etylacetate and Methanol)

Citalopram hydrobromide (100 gm) was dissolved in ethyl acetate (500 ml) and methanol (75 ml) at 55-60° C. then treated with carbon and filtered and washed with ethyl acetate (50 ml). The resulting solution was cooled to 5-10° C., to obtain a crystallized product. The crystallized product was filtered and washed with chilled ethyl acetate to get pure citalopram hydrobromide

### Dry weight=80-90 gm

### f) Citalopram Hydrochloride (Methanollisopropyl Alcohol)

Citalopram hydrochloride (100 gm) was dissolved in methanol (200 ml) at 55-60° C. and then treated with carbon and filtered and washed with methanol (100 ml) The clear filtrate is distilled off completely and diluted with isopropyl alcohol (600 ml). The resulting solution was cooled to 5-10° C., to obtain a crystallized product. The crystallized product was filtered and washed with chilled isopropyl alcohol to get pure citalopram hydrochloride

### Dry weight=85-90 gm

### g) Citalopram Hydrochloride (Etylacetate and Methanol)

Citalopram hydrochloride (100 gm) was dissolved in ethyl acetate (500 ml) and methanol (75 ml)at 55-60° C. and then treated with carbon and filtered and washed with ethyl acetate (50 ml). The resulting solution was cooled to 5-10° C., to obtain a crystallized product. The crystallized product was filtered and washed with chilled ethyl acetate to get pure citalopram hydrochloride

Dry weight=70-75 gm

1. A one pot process for the synthesis of citalopram of the formula 1 starting from 5-cyanophthalide without isolation and purification of the any intermediate stages, which comprises: 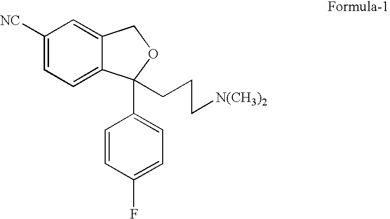
a) subjecting 5-cyanophtalide formula (7) to Grignard reaction with 4-fluorophenyl magnesium bromide in a solvent medium
b) quenching said Grignard reaction mass with aqueous ammonium chloride solution, separating an aqueous layer and an organic layer containing cyanohydroxymethylketone (8), diluting said organic layer with alcoholic solvents and subjecting the resulting solution to a reduction reaction presence of sodium borohydride,
c) diluting the reaction mixture of step (b) with water, and then distilling off low boiling solvent and separating the water immiscible organic solvent
d) subjecting said water immiscible organic solvent containing dihydroxy compound to cyclisation reaction in the presence of catalytic amount of acid,
e) subjecting said cyclized product in a solvent to c-alkylation reaction with 3-N,N′ dimethylamonopropyl chloride in the presence of a strong base to get citalopram.

2. A process as claimed in claim 1, wherein in step a) said 4-fluorophenyl magnesium bromide is reacted with 5-cyanophthalide in a solvent selected from the group consisting of dichloromethane, dichloroethane, chloroform, toluene, benzene and chlorobenzene.

3. A process as claimed in claim 1 wherein said 4-fluorophenyl magnesium bromide is generated in situ by reacting 4-flurobromobenzene with magnesium and a catalytic amount of iodine in tetrahydrofuran medium.

4. A process as claimed in claim 1, wherein in step b), said alcoholic solvent is selected from methanol ethanol and isopropyl alcohol.

5. A process as claimed in claim 1, wherein in step c), after distilling off low boiling solvent, and separation of water immiscible organic solvent, the pH of the reaction mixture is adjusted to 8.5-9.5 using aqueous hydrochloric acid.

6. A process as claimed in claim 5, wherein in step c),said water immiscible solvent is toluene.

7. A process as claimed in claim 6, wherein said dihydroxy compound is subjected to cyclisation in the presence of catalytic amount of acid selected from para toluene sulphonic acid, benzene sulphonic acid and methane sulphonic acid to get 5-cyanophthalane.

8. A process as claimed in claim 1 wherein said C-alkylation reaction of 5-cyanophthalane with N,N dimethylaminopropyl chloride is carried out in the presence of a strong base selected from sodium hydride and, potassium tertiary butoxide in a mixture of DMSO and toluene .

9. A process as claimed in claim 8 wherein after the completion of the c-alkylation reaction, the reaction mass is subjected to acid base work up to get citalopram base starting from 5-cyanophthalide without isolation of any intermediates.

10. A process as claimed in claim 1, wherein i) said Grignard reaction mass of step a) is subjected to a further Grignard reaction with 3N,N′ dimethylaminopropylmagnesium chloride
ii) said Grignard reaction mass of step i) is quenched with aqueous ammonium chloride solution followed by work up to get dihydroxy product
iii) said dihydroxy product is subjected to cyclization in acidic medium to get citalopram directly from 5-cyanophthalide without isolating any intermediate stage.

11. A process as claimed in claim 10, wherein in step ii) said Grignard reaction mass is quenched with aqueous ammonium chloride solution and the organic layer is separated.

12. A process as claimed in claim 11 wherein said separated organic layer is subjected to cyclisation reaction with aqueous acid group selected from acetic acid, hydrochloric acid, sulphuric acid and hydrobromic acid.

13. A process as claimed in claim 12 wherein the said cyclisation reaction pH is adjusted to basic using one or more base such aqueous sodium hydroxide, potassium hydroxide and liquid ammonia solution.

14. A process as claimed in claim 13, wherein the said toluene solution is subjected to acid/base work up followed by concentration under reduced pressure to get citalopram directly from 5-cyanophthalide without isolating any intermediate stages.
